(19) United States
(12) Patent Application Publication
(10) Pub. No.: US 2011/0257464 A1
(43) Pub. Date: Oct. 20, 2011

(54) Electronic Speech Treatment Device Providing Altered Auditory Feedback and Biofeedback

(76) Inventor: Thomas David Kehoe, Boulder, CO (US)

(21) Appl. No.: 12/763,981

(22) Filed: Apr. 20, 2010

(51) Int. Cl. A61F 5/58

(52) U.S. Cl. 600/23

(57) ABSTRACT

A device and method for improving speech. One embodiment comprises an apparatus and method for treating stuttering by providing biofeedback to monitor aspects of the user's voice and also providing altered audio feedback (AAF) to induce fluent speech. Biofeedback features include monitoring phonated intervals. AAF features include delayed auditory feedback (DAF), frequency-altered auditory feedback (FAF), and switching the user's voice between his or her left and right ears multiple times per second. The biofeedback controls AAF parameters including volume, which changes smoothly. Bluetooth is also included to connect to wireless microphones and earphones. Another embodiment comprises an apparatus and method for use by persons without speech disabilities for the purpose of improving public speaking by providing their voice back to their ears altered to appear to be in a different emotional state, thus inducing this preferred emotional state in the person speaking.

### BACKGROUND

### Discussion of Prior Art

Speech is produced by a person releasing air from his or her lungs, then tensing his or her vocal folds, which causes a vibration known as phonation; this humming sound then resonates in the person's pharynx and is articulated into speech sounds by the person's lips, jaw, and tongue to produce vowels and voiced or phonated consonants, e.g., /b/ and /d/. Other speech sounds are voiceless and are produced by releasing air and moving one's articulation muscles without phonation, e.g., /p/ and /t/. To produce the name “Patty,” the voiceless consonant /p/ is produced, followed by the voiced vowel /short-a/, followed by the voiceless consonant /t/, followed by the voiced vowel /long-e/. Thus to say this two-syllable word, one's vocal folds switch on and off several times, resulting in two phonated intervals.

Brain scans of stutterers have found two neurological abnormalities during stuttering: overactivity in the speech motor control area, and underactivity in auditory processing areas “thought to support self-monitoring of speech production.” (Bloodstein, O., Bernstein-Ratner, N. A Handbook on Stuttering, Sixth Edition. Thomson Delmar Learning, 2008, ISBN-13 978-1-4180-4205-5, pages 138-140.)

The speech motor overactivity relates to stutterers overtensing their vocal folds and blocking airflow without producing phonation, as well as overtensing their articulation muscles (lips, jaw, and tongue). Stuttering therapy typically trains stutterers to relax and slow down their speech production muscles, and especially focuses on vocal fold awareness, control, and relaxation. Typically a stutterer is trained to slow down speaking rate by stretching vowels, i.e., by making phonated intervals longer or more consistent. A stutterer may produce phonated intervals in the 10- to 30-millisecond range, and be trained to consistently phonate in 100 millisecond intervals. The resulting speech is slower but with practice can sound clear, natural, and fluent. Training and cognitive effort are required, i.e., the stutterer has to think about both how he is talking and what he is saying.

Stuttering has traditionally been treated without technology, by a speech-language pathologist teaching various techniques to the stutterer. Such techniques fall into two broad categories: speech motor techniques, such as relaxing one's breathing, speaking more slowly by stretching vowels and voiced consonants (i.e., increasing the length of phonated intervals, and reducing articulatory movements of the lips, jaw, and tongue; and psychological techniques for dealing with stressful situations, because stutterers tend to lose control of their speech in stressful situations and are then unable to use the speech motor techniques.

In recent decades various technologies have been developed for treating stuttering. These generally fall into two categories, associated with the two neurological abnormalities associated with stuttering: overactivity in the brain's speech motor control areas, which is treated with biofeedback devices that monitor and display one or more aspects of the stutterer's speech motor activity, such as jaw muscle overactivity, to help the stutterer develop awareness and control of these largely unconscious motor activities; and underactivity in the brain's auditory processing area, especially in the area for self-monitoring of one's speech, which is treated using altered auditory processing (AAF) devices. Traditional stuttering therapy doesn't treat stutterers' auditory processing underactivity, but AAF devices can be used in stressful situations to increase speech motor control, thus reducing the need for teaching stutterers psychological techniques for handling stress.

Biofeedback is the measurement and display of information about a bodily process in real time to help a user improve awareness and control of that bodily process. Speech motor stuttering therapy can be done without biofeedback technology, but is more effective and consistent when the patient receives clear, immediate feedback about parameters of his speech via a biofeedback system. A biofeedback system can also reduce cognitive demands on the user, i.e., amplify awareness so that a process that requires concentration to be aware of, e.g., vocal fold activity, becomes easy to self-monitor while thinking about other things, e.g., the content of one's speech.

The auditory processing underactivity associated with stuttering is treated with altered auditory feedback (AAF). Altered auditory processing devices include delayed auditory feedback (DAF), which delays the user's voice to the user's ear or ears, and frequency-altered auditory feedback (FAF), which changes the pitch of the user's voice in the user's ear or ears. DAF and FAF are widely used to induce fluent speech in stutterers. Each reduces stuttering about 70% (Michelle Lincoln, Ann Packman, Mark Onslow, “Altered auditory feedback and the treatment of stuttering: A review,” Journal of Fluency Disorders, 31, 2006, pages 71-89). The resulting reduction in stuttering occurs without speaking slowly or sounding abnormal, and without training or cognitive effort. The stutterer just puts on headphones and talks without thinking about his speech. DAF and FAF can be combined to increase effectiveness.

Stuttering increases in stressful and noisy situations. Cognitively demanding stuttering therapies that are effective in quiet, low-stress speech clinics break down in noisy, stressful situations. AAF devices can be effective in stressful or noisy situations. Generally, speech therapy that is practiced in stressful situations carries over to less stressful situations, so a stutterer can use an AAF device in a stressful situation to enable him or her to use speech therapy techniques, and then he or she can remove the device in less stressful situations and continue to speak fluently.

U.S. Pat. No. 5,794,203, “Biofeedback system for speech disorders,” issued to Thomas Kehoe on Aug. 11, 1998, and U.S. Pat. No. 6,231,500, “Electronic anti-stuttering device providing auditory feedback and disfluency-detecting biofeedback,” issued to Thomas Kehoe on May 15, 2001, describe machines that combine biofeedback and AAF, configured so that the devices detect disfluent speech in a user who stutters, then provide altered auditory feedback (AAF) to induce the user to speak fluently. The machines switch off the AAF when the user speaks fluently with relaxed speech production muscles. Over time, the user learns to speak fluently and need a device less and less.

Several detection mechanisms were described:
Detection of myoelectric muscle electricity signals indicating overtense speech-production muscles (U.S. Pat. No. 5,794,203). Electromyography (EMG) suffers from two problems: surface electrodes can't pick up vocal fold activity well, and the vocal folds are the most important muscles in stuttering therapy; and users disliked wearing electrodes taped to their faces with many wires connected to a machine.
Analysis of vocal pitch to determine when the user has relaxed his or her speech production muscles, as indicated by a lower vocal pitch, and is less likely to stutter; versus the tense speech production muscles that precede stuttering, as indicated by higher vocal pitch (U.S. Pat. No. 6,231,500). This eliminated the electrodes and picked up vocal fold activity well, but wasn't popular with speech-language pathologists because they are typically trained to teach stutterers to slow their speech by stretching vowels (i.e., eliminating too-short phonated intervals), as opposed to training stutterers to relax their vocal folds to lower their vocal pitch. In general it appears easier for stutterers to speak more slowly than to speak at a lower pitch, although speaking more slowly tends to lower vocal pitch and vice versa, as both result from relaxed speech-production muscles.

These patents describe altering AAF, including DAF and FM, in response to indications that the user was stuttering or speaking fluently. Alterations include changing the delay length (i.e., longer delays when the user is disfluent to force the user to slow down); changing the FAF setting (i.e., increased pitch shifting when the user is disfluent to force the user to relax his or her speech production muscles); and switching AAF on when the user is about to stutter, and off when the user's speech is relaxed and fluent. However, many users prefer to leave the AAF on in one setting, and not use the features that altered the AAF in response to changes in a user's speech. Abrupt changes in how one's hears one's voice, especially having loud AAF switch on and off suddenly, have a jarring effect on the user and increases stress, which is what stutterers try to avoid.

U.S. Pat. No. 5,765,134, titled “Method to electronically alter a speaker's emotional state and improve the performance of public speaking,” invented by Thomas Kehoe, and issued Jun. 9, 1998, described a method for altering a user's voice in the user's ear to change the user's emotional state, while the user is speaking. This invention uses technology similar to the inventions described in U.S. Pat. No. 5,794,203 and U.S. Pat. No. 6,231,500, but instead of being intended for stutterers it was intended for people who don't have a speech disorder but need an assistive device to improve their speech, e.g., when performing public speaking. The device provides the user's voice back to the user's ears, altered to sound like the user is in a different emotional state. This has a powerful effect of inducing the target emotional state in the user. The user can choose from several possible target emotional states:
A state of feeling confident and authoritative can be in induced by shifting the user's voice lower in pitch, adding reverb to sound like the user is speaking in a large auditorium, adding chorus to induce a feeling that the user is not alone, and/or adding a binaural switching effect (in which the user's voice is switched from one ear to the other many times per second) to create a feeling that one's voice is bigger.
A state of feeling happy and enthusiastic, perhaps for performing comedy, can be induced by shifting the pitch of the user's voice higher, enhancing with reverb and chorus, and adding flanging (a ringing sound).
An emotionless tone of voice can be induced with a ring modulator.

These methods were reduced to practice using a portable, battery-powered guitar effects processor (a Zoom 9002), a headset with microphone and headphones, and a second box containing a microphone amplifier and a telephone interface (for altering the emotional state of a user when making telephone calls). The Zoom 9002 was a breakthrough in miniaturization (http://www.zoom.co.jp/english/history/02.php), reducing a 15-pound rack-mounted effects processor to a form that could be carried on a guitar strap, weighing only 10 ounces. Being designed for guitars, not microphones, it needed an external microphone pre-amplifier (another 6 ounces) to be used for vocal processing. The total weight for the Zoom 9002 plus the external microphone pre-amplifier is about one pound. The combination of two devices, two batteries, cables and plugs, etc., make the device cumbersome to use.

U.S. Pat. Nos. 5,794,203; 6,231,500; and 5,765,134 are incorporated herein by reference, in their entirety.

### SUMMARY

To achieve the foregoing and other objects and in accordance with the purpose of the present invention broadly described herein, one embodiment of this invention comprises a system to treat stuttering, with a microphone to receive a user's voice, an accelerometer to detect phonation, connected to a digital signal processor (DSP) to measure the user's phonated intervals, with DSP software to provide AAF including DAF and FAF, with the DSP controlling AAF parameters in accordance with the user's phonated intervals, such as increasing delay length and increasing pitch shift when the user's phonated intervals are too short, and then provided this auditory biofeedback to the user's ears via earphones.

The system could also be used with a Bluetooth wireless microphone and/or earphones.

Another type of AAF switches the user's voice between his or her left and right ears many times per second.

The DSP changes the AAF parameters, including volume, smoothly and continuously, as opposed to clicking the sound on and off.

In another embodiment of the present invention broadly described herein, one embodiment of this invention comprises a system to altering the emotional state of a person who is speaking, for example to improve the performance of public speaking, with the user wearing a microphone and one or more earphones, and a single, self-contained device that alters the user's voice to appear to be in a different emotional state, with a control for the user to select an emotional state (e.g., funny or serious), and another control for the user to select the degree of the emotional state (e.g., very funny vs. just a little funny). This device can also analyze the user's voice and alert the user to undesirable vocal characteristics, e.g., microtremors indicating stress or lying.

### DETAILED DESCRIPTION

The present Invention comprises several improvements over U.S. Pat. Nos. 5,794,203 and 6,231,500 to provide an apparatus and method for combining biofeedback and AAF. These improvements include:
Measuring phonated intervals to detect dysfluent speech. The user wears an accelerometer on his or her throat to detect vocal fold vibrations, or the phonation that produces vowels and voiced consonants. A computer measures the length of these phonated intervals, interspersed with voiceless consonants, silent dysfluencies or blocks, and not talking. Too-short phonated intervals precede stuttering so the user is trained to slow his or her speech by stretching vowels, i.e., to make these phonated intervals consistent and a little longer. The machine alerts the user when his or her phonated intervals are too short. This type of biofeedback fits well with the training most speech-language pathologists received in graduate school, and requires no electrodes.
Using a new type of AAF to switch the sound of the user's voice in headphones between the user's left and right ears many times per second. This can be combined with DAF and FAF to increase effectiveness.
Fading the AAF sound volume up and down smoothly. In U.S. Pat. Nos. 5,794,203 and 6,231,500 AAF sound switched on and off-abruptly. This is unpleasant to the ears and some users complained of a “loose wire” in their machines switching sound on and off (not realizing that the machine was analyzing their speech and switching AAF on and off in response to fluent or dysfluent speech).
Using an accelerometer worn on the user's throat to trigger a switch to turn the machine on when the user speaks and off when the user stops speaking. The use of an accelerometer is more effective than a microphone for detecting speech (Fredric Lindstrom, Keni Ren, Haibo Li, Kerstin Persson Waye, “Comparison of Two Methods of Voice Activity Detection in Field Studies,” Journal of Speech, Language, and Hearing Research, Vol. 52, pages 1658-1663, December 2009) and is more useful when making a voice-operated switch to switch a machine on when the user speaks and off when the user stops speaking. U.S. Pat. No. 5,590,241, issued to Park et al. on Dec. 31, 1996, describes a combination of an accelerometer and microphone for the purpose of enhancing speech signals in a noisy environment. In accordance with the present invention, both an accelerometer and a microphone may be worn in a single neckband and used to switch a machine on when the user speaks and pick up the user's voice.
Bluetooth technology enables users to wear wireless earphones, or a wireless earset combining a microphone and one or more earphones.
The apparatus may be a self-contained device, which can include all signal processing functions, including amplification and digital signal processing (DSP), and needs only an external microphone and earphones. Alternatively, the signal processing functions may be implemented in software to run on a laptop computer or an Apple iPhone or similar device, again needing only an external microphone and earphones. This represents an improvement over the device described in U.S. Pat. No. 5,765,134.
Detecting vocal microtremors that indicate stress and providing biofeedback to the user that he or she appears to be lying, the user can use techniques to calm his or her voice and appear more sincere to listeners. Similar detection systems have been used to detect stress associated with lying, such as for law enforcement purposes.

To measure phonated intervals, the user wears a vibration sensor on his or her throat. The vibration sensor consists of a single-axis accelerometer with a minimum bandwidth of 80-300 Hz; e.g., Model ACH-01-04 made by Measurement Specialties, Inc. 1000 Lucas Way Hampton, Va. 23666. The vibration sensor is worn with an elastic band paralateral to midline and just inferior to the thyroid prominence. An analog vibration sensor can be connected to a signal-conditioning amplifier (e.g., Model IB-ACH-01 Amplifier Box, made by Measurement Specialties) that is then connected to a computer, or a USB digital vibration sensor can be connected directly to a computer.

The computer receives data from the vibration sensor indicating either vibration or lack of vibration. Computer software measures the length (time duration) of the vibrations, i.e., the phonated intervals. The computer monitor displays a bar graph, with bars typically ranging from 30 milliseconds to 200 milliseconds, but as wide a range as 10 milliseconds to 1000 milliseconds. Each phonated interval is measured and added to its appropriate bar. The bar graphs may be color-coded according to the durations of phonated intervals, for example, with 10 to 50 milliseconds in red on the left, and 50 to 100 milliseconds in yellow in the middle, and 100 milliseconds and longer in green on the right. The user then tries to make the green graphs tall while keeping the red and yellow graphs short, by speaking with consistently long phonated intervals.

The user also wears a microphone. This can be a throat microphone conveniently worn in the same elastic band with the vibration sensor, and clearly picking up the user's phonation, to help the user develop vocal fold awareness and control. Alternatively, the user can wear any other type of microphone.

The microphone signals are transferred into the computer, where the user's voice is then digitized and processed. The transfer may be via an electrical cable plugged into the computer or via wireless transmission. Audio Units software make AAF computer software easy to develop. According to Wikipedia, “Audio Units (AU) are a system-level plug-in architecture provided by Core Audio in Mac OS X developed by Apple Computer. Audio Units are a set of application programming interface services provided by the operating system to generate, process, receive, or otherwise manipulate streams of audio in near-real-time with minimal latency.” (Wikipedia, “Audio Units,” accessed 2010 Apr. 16.)

In particular, AUDelay produces delayed auditory feedback (DAF), AUPitch produces frequency-altered auditory feedback (FAF), and AUMixer mixes the DAF and FAF into a combined signal that is provided to AudioDeviceOutput, which then outputs to the user's headphones. AudioDeviceOutput also processes the sound from the microphone (despite its name, it handles both input and output).

AUDynamicsProcessor can be connected to the software that measures and counts phonated intervals, with parameters such as attack and release times controlled, to make the AAF volume change smoothly in response to the user's vocal characteristics such as phonated intervals or vocal frequency.

Other companies make software similar to Audio Units that are easily plugged into a software application for use in accordance with the present invention. Some of these competing technologies are for Apple's Mac OS X operating system and provide functions not provided by Apple's Audio Units, e.g., Blue Cat's Stereo Chorus. Others provide similar functionality for other operating systems, e.g., LADSPA, DSSI and LV2 for Linux; and Microsoft's DirectX for Windows computers.

The AAF can be provided to the user's headphones via either a cable or wireless transmission. Many laptop computers now include Bluetooth support for wireless microphones and earphones.

In an alternative embodiment, instead of connecting to a computer, the accelerometer, microphone, and earphones connect to a self-contained portable device that includes a digital signal processor (DSP) such as a Texas Instruments C5515 or an Analog Devices Blackfin SHARC. The DSP chips are capable of monitoring input from an accelerometer, receiving a speech signal from a microphone, altering the speech signal with DAF, FAF or other types of AAF, providing biofeedback functions, and outputting an altered audio signal to an amplifier and headphones. A Bluetooth wireless interface to a microphone or earphones can be provided on a single chip such as the BlueCore made by CSR PLC (Cambridge, England), or the LMX9838 made by National Semiconductor (Santa Clara, Calif.). Such a self-contained portable device needs only a power supply and battery, a small screen and buttons to drive menus, and jacks for the input and output (microphone, earphones, etc.) in addition to the DSP and Bluetooth chips.

U.S. Pat. No. 5,765,134 describes only using altered auditory feedback (AAF) to alter a user's emotional state while speaking. Today, it is possible to also analyze a user's voice to detect signs of stress, and provide this information back to the user in real-time (a form of biofeedback). Digital Voice Stress Analysis (D-VSA) software applications are available from several companies, including NITV-CVSA; Dektor-PSE5128, Diogenes-Lantern and Baker-FVSA. These software applications detect microtremors in the muscles that control the larynx or vocal folds. Some claim, controversially, that such microtremors show that a speaker is lying. Some of these software applications are expensive and intended for law enforcement personnel; others are for amateurs, such as the free Skype app from KishKish, or the cellphone service LiarCard.

It is uncontroversial that microtremors indicate vocal stress. Another way to measure vocal stress is with frequency analysis, as a lower vocal pitch indicates lower speech motor activity. Frequency analysis is also easily implemented in software.

### OPERATION

Treatment of stuttering with this device starts with a stutterer working with a speech-language pathologist. In the speech clinic, the speech-language pathologist uses a computer-based embodiment of the device. The stutterer wears an elastic neckband with an accelerometer and microphone, both plugged into the computer. He or she also wears headphones plugged into the computer. He or she sees on the computer screen a bar graph with perhaps 12 vertical bars, representing phonated intervals varying from short on the left and displayed in red, to long on the right and displayed in green, and medium in the middle displayed in yellow. He or she also sees a visual display of vocal pitch.

The speech-language pathologist trains the stutterer to slow his or her speech by stretching vowels and voiced consonants, and relaxing his or her speech production muscles. When the stutterer hits these targets, he or she sees the green bars on the right growing in height, and the red bars on the left not growing or shrinking. He or she also sees his or her vocal pitch displayed as low.

The speech-language pathologist and stutterer begin with simple vowel sounds, then go to single-syllable words, multi-syllable words, phrases, sentences, and then multi-sentence reading, monologues, and conversation.

The speech-language pathologist and stutterer begin with AAF on all the time (continuous mode). DAF is used to induce a slower speaking rate, and an FAF downshift is used to induce slow speech with relaxed speech-production muscles. As the stutterer masters the speech motor targets the AAF is switched to biofeedback mode, in which the AAF parameters and volume change as the user's speech changes. If the user misses targets and produces too-short phonated intervals, and/or his or her vocal pitch is too high, he or she hears his or her voice in the headphones delayed and shifted down in pitch. But as he or she hits the speech motor targets the delay becomes shorter, the pitch shift diminishes, and the volume in the headphones diminishes.

After some time (a few hours for most stutterers) the stutterer is able to hit the speech motor targets consistently, holding a conversation with the speech-language pathologist while speaking with slow, relaxed, fluent speech.

Most stutterers can then use a small, portable device outside of the speech clinic. The portable device has a smaller display, perhaps showing only six bars representing phonated intervals.

The stutterer may wear the same elastic throat band but replace the headphones with Bluetooth wireless earphones or hearing aid-style FM wireless receivers. Or the stutterer may choose not to wear the elastic throat band and instead wear only a Bluetooth cellphone earset with microphone and earphone built in.

The stutterer then wears the device for most of the day, especially in stressful situations. At first he or she has to concentrate on his or her speech, hearing the AAF and watching the biofeedback graphical display. But over time he or she learns to speak fluently with little or no cognitive effort, in more and more stressful situations. Over time he or she needs the device less and less, until he or she uses it only for very stressful situations such as public speaking.

Severe stutterers may have to speak abnormally slowly at first, with the delay set at over 100 milliseconds, phonated intervals at least 150 milliseconds, and a speaking rate five times slower than normal speech. This, however, is an improvement over stuttering that is unclear, physically exhausting, and more than ten times slower than normal speech. A severe stutterer may return to the speech clinic several times to work with a speech-language pathologist and practice on the computer-based device to get his fluent speech closer to normal-sounding speech before trying the portable device “solo” outside the speech clinic.

Another embodiment of the present invention comprises a public speaking aid that can be used by a person with normal speech, but who may have a fear of public speaking or may just want to make a more effective presentation. In one embodiment, the person downloads an iPhone app and plugs in a combination microphone and earphones to his or her iPhone.

The iPhone app displays a user interface with a simulated two-position slide switch and a simulated slider. The user moves the slide switch to either “Funny” or “Serious.” He or she then moves the slider on a ten-point scale from “Mild” to “Extreme.”

E.g., he or she starts his or her presentation with a joke, so switches to “Funny.” He or she is nervous so sets the slider at “Extreme.” The joke gets a big laugh, and the speaker switches the app to “Serious,” with the slider in the middle at 5, and delivers the rest of the presentation.

In the “Happy” setting, the speaker hears the pitch of his or her voice shifted upward. At the “Mild” setting this is 0.1 octaves up. Each point on the scale increases the pitch shift one-tenth octave, with “Extreme” providing a one-octave pitch shift.

The “Happy” setting also includes reverb, beginning with a subtle echo at the “Mild” setting” to sounding like you're speaking in a vast cathedral at the “Extreme” setting.

The “Happy” setting also includes a chorus effect and the binaural switching effect (in which the user's voice is switched between his or her left and right ears typically 24 times per second). These effects can be provided at a lower volume (compared to the volume of the other effects) at the “Mild” setting, and at full volume at the “Extreme” setting.

When the user switches the app to the “Serious” mode, he or she hears his or her voice shifted lower in pitch, again with the slider setting the degree of pitch shift from “Mild” to “Extreme.” The “Serious” mode has reverb, chorus, and the binaural switching effect similar to the “Happy” setting.

The “Serious” setting also has a delay (delayed auditory feedback, or DAF) feature, on a second simulated slider. The user selects his or her speaking rate, from “Normal” to “Very Slow.” “Normal” is no delay (0 milliseconds), and “Very Slow” is 200 milliseconds. DAF forces the user to speak slowly, which makes the user seem more confident and authoritative while simultaneously making the user's voice clearer to listeners whose native language isn't the native language of the speaker, or for persons with hearing impairments.

The user can also switch on a feature to detect microtremors, indicating vocal stress or the appearance of lying. If the app detects microtremors during the presentation, the user hears a series of tones, with more tones and/or higher pitch indicating more microtremors. He or she then pauses, takes a breath, relaxes and starts again, sounding more sincere.

### CONCLUSIONS, RAMIFICATIONS, AND SCOPE

Thus the reader will see that at least one embodiment of the speech treatment device provides a more effective yet more convenient and comfortable treatment for speech disorders, such as stuttering, or to improve the speech of persons without speech disorders, such as persons performing public speaking.

While my above description contains many specificities, these should not be construed as limitations on the scope, but rather as an exemplification of several embodiments thereof. Many other variations are possible.

Accordingly, the scope should be determined not by the embodiments illustrated, but by the appended claims and their legal equivalents.

I claim:

1. A system to treat stuttering, comprising:
means for receiving a user's voice,
means for detecting phonation in the user's voice;
means for measuring interval durations of the user's phonation;
means for using said phonation duration intervals to control parameters of altered auditory feedback; and
means for providing said altered auditory feedback to the user.

2. The system of claim 1, wherein said means for receiving the user's voice comprises a microphone.

3. The system of claim 1, wherein said means for detecting the user's phonation comprises an accelerometer.

4. The system of claim 1, wherein said means for measuring phonation interval durations comprises a digital signal processor (DSP).

5. The system of claim 1, with means to visually inform the user of said phonation interval durations.

6. The system of claim 1, wherein said means for using said phonation duration intervals to control parameters of altered auditory feedback comprises a digital signal processor (DSP).

7. The system of claim 1, wherein said means for providing altered auditory feedback to the user comprises earphones.

8. The system of claim 1, wherein said means for providing altered auditory feedback to the user provides a Bluetooth connection to at least one device selected from the group consisting of microphones, earphones, and combinations thereof.

9. The system of claim 1, wherein said means for providing altered auditory feedback is operative to provide altered auditory feedback selected from delayed auditory feedback, frequency-altered auditory feedback, and combinations thereof.

10. The system of claim 9, wherein said means for controlling altered auditory feedback is operative to change the delay length of said delayed auditory feedback in relation to said phonated interval durations.

11. The system of claim 9, wherein said means for controlling altered auditory feedback is operative to change the pitch of said frequency-altered auditory feedback in relation to said phonated interval durations.

12. The system of claim 1, wherein said means for controlling altered auditory feedback is operative to change the volume of said altered auditory feedback in relation to said phonated interval durations.

13. The system of claim 1, wherein said means for providing altered auditory feedback cyclically increases and decreases the volume of the user's voice in one ear while simultaneously decreasing and increasing in the volume in the user's other ear.

14. A system to treat stuttering, comprising:
biofeedback means for monitoring aspects of a user's speech; and
means for providing altered auditory feedback to a user;
wherein one or more parameters of said altered auditory feedback are controlled by said biofeedback means based on said monitored speech aspects, with changes in said one or more altered auditory feedback parameters made in a manner selected from the group of smoothly and non-discontinuously.

15. The system of claim 14, wherein one of said altered auditory feedback parameters is volume.

16. A system for altering the emotional state of a person who is speaking, comprising:
a transducer for converting a user's voice into an audio signal;
a self-contained processor for altering said audio signal; and
at least one transducer for converting said altered audio signal into an altered voice and providing said altered voice to the user to enable the user to hear his or her altered voice;
wherein said processor is operative to alter said audio signal in a manner that when said altered voice is provided to the user, the user hears his or her voice altered in one or more ways that induce an emotional state selected by the user.

17. The system of claim 16, wherein said self-contained processor alters said audio signal to alter the pitch of the user's voice in one or more of the user's ears.

18. The system of claim 16, wherein said self-contained processor alters said audio signal to delay said user's voice to one or more of said user's ears.

19. The system of claim 18, wherein said self-contained processor is operative to delay said audio signal by repeating and diminishing said delayed audio signal in an echo pattern.

20. The system of claim 16, wherein said self-contained processor is operative to mix said audio signal with one or more delayed, pitch-modulated copies of itself to achieve a chorus effect.

21. The system of claim 16, wherein said self-contained processor is operative to mix two identical audio signals together, with one audio signal time-delayed by a small and gradually changing amount, to produce a swept comb filter effect with peaks and notches produced in the resultant frequency spectrum, related to each other in a linear harmonic series, to achieve a flanging effect.

22. The system of claim 16, wherein said self-contained processor is used with two transducers to provide said altered voice to the user's two ears, while alternately and cyclically increasing and decreasing the volume of said altered voice in one ear of the user while simultaneously decreasing and increasing the volume in the user's other ear.

23. The system of claim 16, wherein said self-contained processor is operative to analyze characteristics of said audio signal and provide information to the user about corresponding characteristics of his or her voice.

24. The system of claim 23, wherein said self-contained processor is operative to detect the presence of microtremors in the user's voice.

25. The system of claim 23, wherein said self-contained processor is operative to analyze the pitch of the user's voice and provide a display indicating the user's vocal pitch.

26. A method to treat stuttering, comprising the steps of:
detecting a user's phonation;
measuring interval durations of said phonation;
informing the user of said durations;
providing altered auditory feedback to the user; and
using said durations of said intervals to control parameters of said altered auditory feedback.

27. The method of claim 26, wherein said detecting step comprises using an accelerometer.

28. The method of claim 26, wherein said providing step comprises providing altered auditory feedback selected from delayed auditory feedback, frequency-altered auditory feedback, and combinations thereof.

29. The method of claim 28, wherein said providing step comprises delaying said delayed auditory feedback (DAF) by an amount related to said durations of phonation.

30. The method of claim 28, wherein said providing step comprises pitch shifting said frequency-altered auditory feedback by an amount related to said durations of phonation.

31. The method of claim 26, wherein providing step comprises changing volumes of said altered auditory feedback in relation to said durations of phonation.
